(19) United States
(12) Patent Application Publication
(10) Pub. No.: US 2025/0268618 A1
(43) Pub. Date: Aug. 28, 2025

(54) ULTRASONIC ABLATION CATHETER AND ULTRASONIC ABLATION DEVICE

(71) Applicant: SHANGHAI GOLDEN LEAF MED TEC CO., LTD., Shanghai (CN)

(72) Inventors: Hongguang CAO, Shanghai (CN); Liang JI, Shanghai (CN); Meijun SHEN, Shanghai (CN)

(73) Assignee: SHANGHAI GOLDEN LEAF MED TEC CO., LTD., Shanghai (CN)

(21) Appl. No.: 19/086,130

(22) Filed: Mar. 21, 2025

Related U.S. Application Data
(63) Continuation of application No. PCT/CN2023/109723, filed on Jul. 28, 2023.

(30) Foreign Application Priority Data
Sep. 21, 2022 (CN) 202211153107.0
Sep. 21, 2022 (CN) 202222508634.0

(51) Int. Cl. A61B 17/32; A61B 17/00

(52) U.S. Cl. CPC A61B 17/320068; A61B 17/00234; A61B 2017/00238; A61B 2017/00305; A61B 2017/00477; A61B 2017/00862; A61B 2017/320069

(57) ABSTRACT

Disclosed are an ultrasonic ablation catheter and an ultrasonic ablation device. The ultrasonic ablation catheter comprises: a central cavity tube, provided with a central hole formed in the axial direction of the central cavity tube; a central wire, provided with at least one ultrasonic transducer, a first end of the central wire being movably arranged through the central hole and being switchable between a contracted state and an extended state; and an elastic support pre-formed into a preset shape and sheathing the central wire, a first end of the elastic support being movably arranged through the central hole and being switchable between a compressed state and an expanded state.

### BACKGROUND

### Technical Field

The present disclosure relates to an ultrasonic ablation catheter, also relates to an ultrasonic ablation device including the ultrasonic ablation catheter, and relates to the technical field of medical instruments.

### Related Art

Ultrasonic energy has a good directionality and penetrability. Compared with radio frequency energy, ultrasonic ablation uses lower energy and do not rely on tissue conduction for energy transfer, so that damage to intima tissue of blood vessels can be avoided in a treatment process. Therefore, the ultrasonic energy may be one of ideal energy sources in the field of ablation treatment.

Currently, a basic construction of an ultrasonic transducer includes a piezoelectric sheet, an electrode, a backing, and a matching layer. By using the backing, the ultrasonic transducer controls a frequency and an impulse response of the ultrasonic transducer, and controls directionality of energy propagation of the ultrasonic transducer. Due to the fact that a high-damping and high-attenuation material is usually used for the backing of the ultrasonic transducer, part of ultrasonic energy injected into the backing is converted into thermal energy, causing energy loss. This not only reduces the electroacoustic conversion efficiency of the ultrasonic transducer, but also causes undesirable consequences in human body tissue. For example, in a blood vessel, an excessively hot transducer surface may damage intima of a treatment region, causing vascular stenosis or even occlusions to a treatment object in a long term. Further for example, in blood, an ultrasonic element having an increased surface temperature may cause the blood to clot and agglomerate on the surface of the transducer, which hinders release of ultrasonic energy, further reduces the electroacoustic conversion efficiency of the ultrasonic transducer, and even causes damage to the transducer. In addition, the forming of the surface clot increases the risk of occurrence of a clot embolism.

### SUMMARY

The first technical problem to be resolved by the present disclosure is to provide an ultrasonic ablation catheter, so as to improve the safety of ultrasonic ablation treatment.

Another technical problem to be resolved by the present disclosure is to provide an ultrasonic ablation device including the ultrasonic ablation catheter.

To achieve the foregoing objective, the present disclosure adopts the following technical solution.

According to a first aspect of embodiments of the present disclosure, an ultrasonic ablation catheter is provided, including:
a central cavity tube, a central hole being provided along an axial direction of the central cavity tube;
a central wire, the central wire being provided with at least one ultrasonic transducer, and a first end of the central wire movably passing through the central hole, to switch between a contracted state and an extended state; and
a resilient stent, the resilient stent being preformed into a set shape and being nested on the central wire, and a first end of the resilient stent movably passing through the central hole, to switch between a compressed state and an expanded state, where
when the central wire is in the contracted state, the ultrasonic transducer and a second end of the resilient stent are accommodated in the central cavity tube together with a second end of the central wire, for the resilient stent to be in the compressed state; and when the central wire is in the extended state, the ultrasonic transducer and the second end of the resilient stent extend out of the central cavity tube together with the second end of the central wire, for the resilient stent to be in the expanded state and restore to the set shape.

Preferably, the resilient stent is formed into a mesh shape by intertwining a plurality of stent threads.

Preferably, the resilient stent includes a plurality of wrap parts, a plurality of ultrasonic transducers are mounted on the central wire, and the wrap parts are respectively wrapped around outer sides of the ultrasonic transducers.

Preferably, the resilient stent includes a plurality of wrap parts, a plurality of ultrasonic transducers are mounted on the central wire, and the plurality of wrap parts and the plurality of ultrasonic transducers are arranged crosswise along the axial direction of the central cavity tube, for any two adjacent ultrasonic transducers to have one of the wrap parts therebetween.

Preferably, a balloon is further wrapped around an outer side of the ultrasonic transducer, and the balloon is arranged between the resilient stent and the ultrasonic transducer.

Preferably, a cooling pipe is disposed in the central hole, a first end of the cooling pipe is configured to be connected to a cold medium supply apparatus, and a second end of the cooling pipe extends out of the central cavity tube, to correspond to a position of the ultrasonic transducer when the central wire is in the extended state.

Preferably, a first channel and a second channel communicated with each other are provided in the central wire, the first channel is configured to be communicated with a liquid outlet of the cold medium supply apparatus, and the second channel is configured to be communicated with a liquid inlet of the cold medium supply apparatus, to jointly form a cooling circulation loop; and a cold medium periodically flows in the cooling circulation loop, to cool the ultrasonic transducer.

Preferably, the ultrasonic transducer is mounted on an outer side of the central wire by pasting and/or embedding, and a width of the ultrasonic transducer is less than a diameter of the central wire, to partially cover the central wire; and the central wire is capable of rotating around an axial direction of the central wire in 360°, to drive the ultrasonic transducer to rotate in 360°.

Preferably, the ultrasonic ablation catheter further includes a connecting member, the connecting member being connected and fixed to the second end of the resilient stent and the second end of the central wire.

Preferably, the resilient stent is provided with a transparent refractive region and an opaque closed region, and the transparent refractive region corresponds to the ultrasonic transducer, to refract ultrasonic waves emitted by the ultrasonic transducer.

According to a second aspect of the embodiments of the present disclosure, an ultrasonic ablation device is provided, including the ultrasonic ablation catheter.

Compared with the existing technology, according to the ultrasonic ablation catheter and device provided in the present disclosure, controllable ablation is performed by using the ultrasonic transducer mounted on the central wire, so as to improve the ablation effect on pathological tissue. In addition, the resilient stent in the expanded state may distract a blood vessel, to prevent the ultrasonic transducer from being in contact with a blood vessel wall, thereby preventing damage to the blood vessel wall. Besides, the ultrasonic transducer may alternatively be cooled by infusing cold saltwater or a cold gas, thereby further improving the safety of an ablation operation.

### BRIEF DESCRIPTION OF THE DRAWINGS

FIG. 1 is a schematic structural diagram of an ultrasonic ablation catheter according to a first embodiment of the present disclosure;

FIG. 2 is a schematic structural diagram of an ultrasonic ablation catheter according to a second embodiment of the present disclosure;

FIG. 3 is a schematic structural diagram of an ultrasonic ablation catheter according to a third embodiment of the present disclosure;

FIG. 4 is a schematic structural diagram of an ultrasonic ablation catheter according to a fourth embodiment of the present disclosure;

FIG. 5 is a schematic structural diagram of an ultrasonic ablation catheter according to a fifth embodiment of the present disclosure;

FIG. 6 is a schematic structural diagram of an ultrasonic ablation catheter according to a sixth embodiment of the present disclosure;

FIG. 7 is a schematic structural diagram of a connection between a central wire and a cold medium supply apparatus according to the first embodiment of the present disclosure; and

FIG. 8 is a schematic structural diagram of a connection between a central wire and a cold medium supply apparatus according to a seventh embodiment of the present disclosure.

### DETAILED DESCRIPTION

The technical content of the present disclosure will be described in detail below with reference to the accompanying drawings and specific embodiments.

### First Embodiment

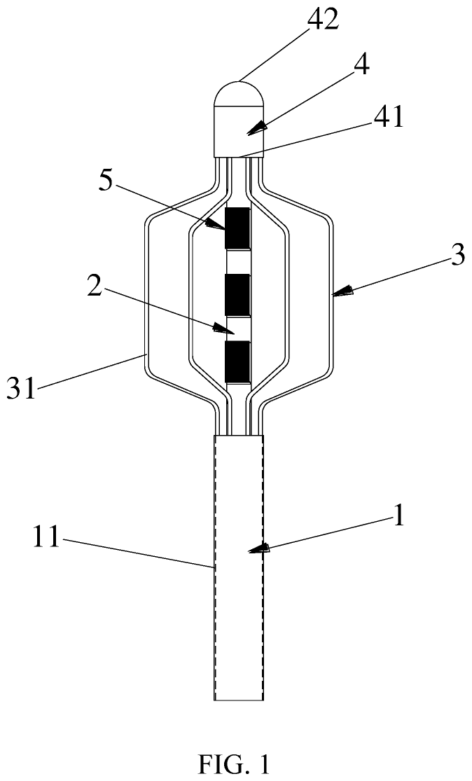

Referring to FIG. 1, this embodiment of the present disclosure provides an ultrasonic ablation catheter, including: a central cavity tube 1, a central wire 2, a resilient stent 3, a connecting member 4, and an ultrasonic transducer 5.

Specifically, in this embodiment, a central hole 11 is provided along an axial direction of the central cavity tube 1. That is, in this embodiment, the central cavity tube 1 is a hollow circular tube open at two ends, and the central hole 11 is configured to provide an accommodating space for the central wire 2 and the resilient stent 3.

At least one ultrasonic transducer 5 is mounted on the central wire 2. The ultrasonic transducer 5 is configured to generate ultrasonic waves, so that after the ultrasonic ablation catheter reaches a site to be ablated, energy is gathered to a sufficient intensity at a particular region where the ultrasonic waves are focused, to enable a focal region to reach a high instantaneous temperature, to destroy pathological tissue in the particular region, thereby achieving the purpose of an ablation operation. Specifically, in this embodiment, three ultrasonic transducers 5 are provided, and the three ultrasonic transducers 5 are disposed at intervals along a length direction of the central wire 2, to improve the energy focusing efficiency.

The central wire 2 is a cylindrical metal wire made of a NiTi alloy material, so that the central wire 2 has a particular degree of rigidity. A first end (that is, the end away from the connecting member 4 in FIG. 1) of the central wire 2 movably passes through the central hole 11, so that the central wire 2 can be switched between a contracted state and an extended state. Specifically, when a second end (that is, the end close to the connecting member 4 in FIG. 1) of the central wire 2 is entirely accommodated in the central hole 11, the central wire 2 is in the contracted state. In this case, the three ultrasonic transducers 5 mounted on the central wire 2 are also accommodated in the central hole 11 together with the second end of the central wire 2. When the second end (that is, the end close to the connecting member 4 in FIG. 1) of the central wire 2 extends out of the central hole 11 by a first set distance (the first set distance may be set by users according to actual needs: for example: 10 mm to 60 mm), the central wire 2 is in the extended state. In this case, all of the three ultrasonic transducers 5 extend out of the central hole 11, for ultrasonic ablation use.

The resilient stent 3 is preformed into a set shape, and is nested on the central wire 2. Specifically, the resilient stent 3 includes a plurality of stent threads 31 circularly distributed around a central axis of the central wire 2. The stent threads 31 are mostly NiTi threads, and insulation treatment is carried out on surfaces of the NiTi threads. A length direction of each stent thread 31 extends along a length direction of the central cavity tube 1. Two ends of all of the stent threads 31 form a first end and a second end of the resilient stent 3, respectively. In addition, middle sections of all of the stent threads 31 are bent to present a U shape, to form a petal-shaped resilient stent 3.

Referring to FIG. 1, a first end of the petal-shaped resilient stent 3 is away from the connecting member 4, a second end of the petal-shaped resilient stent 3 is close to the connecting member 4, and a first end of the petal-shaped resilient stent 3 movably passes through the central hole 11, to switch between a compressed state and an expanded state. Specifically, when the second end of the petal-shaped resilient stent 3 is entirely accommodated in the central hole 11, the petal-shaped resilient stent 3 is in the compressed state, that is, the middle sections of all of the stent threads 31 are compressed in the central hole 11, to facilitate entry of the ultrasonic ablation catheter into a human blood vessel. When the second end of the petal-shaped resilient stent 3 extends out of the central hole by a second set distance (the second set distance may be set by users according to actual needs: for example: 10 mm to 60 mm), the petal-shaped resilient stent 3 is in the expanded state. In this case, the middle sections of all of the stent threads 31 restore to the set shape outside the central cavity tube 1, to block a blood vessel wall and the ultrasonic transducer 5, so as to prevent the ultrasonic transducer 5 from being in direct contact with the blood vessel wall and causing damage to the blood vessel.

In addition, it may be understood that, in the foregoing embodiment, when the central wire 2 is in the contracted state, the ultrasonic transducer 5 and a second end of the resilient stent 3 are accommodated in the central cavity tube 1 together with a second end of the central wire 2, for the resilient stent 3 to be in the compressed state. When the central wire 2 is in the extended state, the ultrasonic transducer 5 and the second end of the resilient stent 3 extend out of the central cavity tube 1 together with the second end of the central wire 2, for the resilient stent 3 to be in the expanded state and restore to the set shape. That is, in this embodiment, movements of the central wire 2, the resilient stent 3, and the ultrasonic transducer 5 are synchronous. That is, the first set distance is equal to the second set distance. A control handle may be mounted at the end of the central cavity tube 1 away from the connecting member 4, and working forms of the central wire 2, the resilient stent 3, and the ultrasonic transducer 5 are controlled by the control handle.

The connecting member 4 is a hollow circular tube open at one end. Referring to FIG. 1, the end of the connecting member 4 close to the central cavity tube 4 is an open end 41, and the end away from the central cavity tube 4 is a closed end 42. The connecting member 4 is configured to fix the central wire 2 and the second end of the resilient stent 3. When the central wire 2 and the resilient stent 3 are pulled along the axial direction of the central cavity tube 1 by using the control handle, the open end 41 of the connecting member 4 gradually approaches the central cavity tube 1, until the open end 41 of the connecting member 4 abuts against an end surface of the central cavity tube 1, the central wire 2 is in the contracted state, and the resilient stent 3 is in the compressed state. When the central wire 2 and the resilient stent 3 are pushed along the axial direction of the central cavity tube 1 by using the control handle, the open end 41 of the connecting member 4 gradually gets away from the central cavity tube 1, until the connecting member 4 moves by the first set distance, the central wire 2 is in the extended state, and the resilient stent 3 is in the expanded state.

In an embodiment of the present disclosure, a cooling pipe is disposed in the central hole 11, a first end of the cooling pipe is configured to be connected to a cold medium supply apparatus, and a second end of the cooling pipe extends out of the central cavity tube 1, to correspond to a position of the ultrasonic transducer 5 when the central wire 2 is in the extended state. Specifically, in this embodiment, the cooling pipe may be a cold saltwater injection pipe or a cold gas injection pipe. Correspondingly, the cold medium supply apparatus may be a cold saltwater supply apparatus or an apparatus for supplying a low-temperature gas, such as carbon dioxide. After the ultrasonic ablation catheter extends into a human body blood vessel, cold saltwater or a cold gas may be provided through the cooling pipe, to cool the ultrasonic transducer 5, so as to further avoid damage caused to a blood vessel wall by a high-temperature ultrasonic transducer 5, thereby improving the safety of an ablation operation.

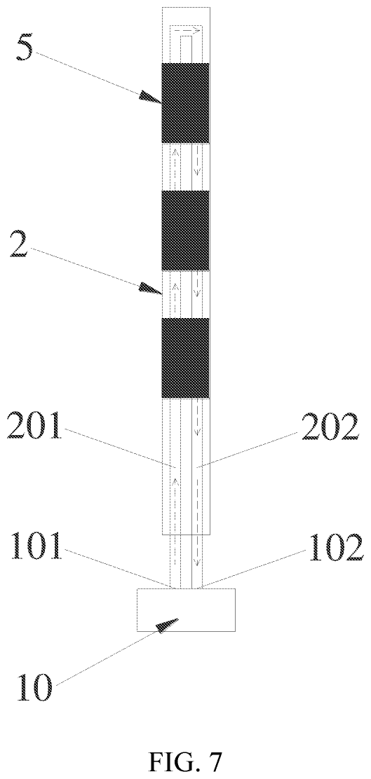

As shown in FIG. 7, in another embodiment, a first channel 201 and a second channel 202 communicated with each other are provided in the central wire 2. The ultrasonic transducer 5 is nested on the central wire 2, to surround around an outer side of the central wire 2 in 360°. Moreover, in this embodiment, both the first channel 201 and the second channel 202 pass through a bottom portion of the central wire 2 and do not pass through a top portion of the central wire 2. The first channel 201 is configured to be communicated with a liquid outlet 101 of a cold medium supply apparatus 10, and the second channel 202 is configured to be communicated with a liquid inlet 102 of the cold medium supply apparatus 10, to jointly form a cooling circulation loop (the loop shown by the dashed arrow in FIG. 7). A cold medium is periodically introduced into the cooling circulation loop, and the ultrasonic transducer 5 can be cooled by circulating flow of the cold medium in the central wire 2, to further avoid damage caused to a blood vessel wall by a high-temperature ultrasonic transducer 5, thereby improving the safety of an ablation operation. It may be understood that, in this embodiment, the cold medium periodically circulates only in the cooling circulation loop, and does not leak out to a patient body, so as to use internal cooling to avoid interference to the operation.

In an embodiment of the present disclosure, the central cavity tube 1 is a serpentine tube, so that the central cavity tube 1 has a controllable bending function, thereby further improving the use convenience of the ultrasonic ablation catheter.

In conclusion, according to the ultrasonic ablation catheter provided in this embodiment of the present disclosure, controllable ablation is performed by using the ultrasonic transducer 5 mounted on the central wire 2, so as to improve an ablation effect on pathological tissue. In addition, the resilient stent 3 in the expanded state may distract a blood vessel, to prevent the ultrasonic transducer 5 from being in contact with a blood vessel wall, thereby preventing damage to the blood vessel wall. Besides, the ultrasonic transducer 5 may alternatively be cooled by infusing cold saltwater or a cold gas, thereby further improving the safety of an ablation operation.

### Second Embodiment

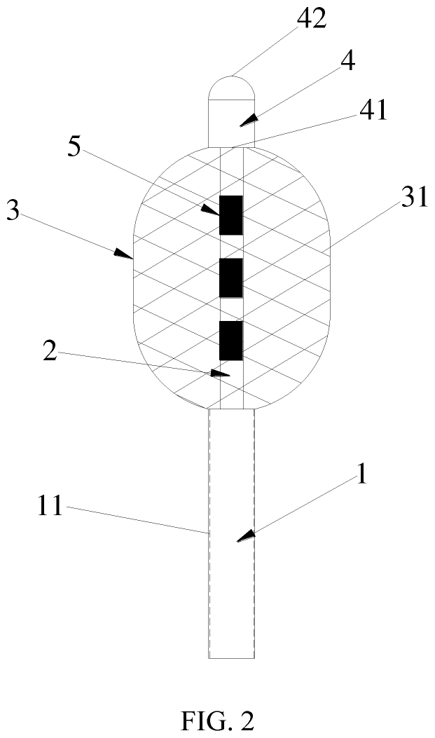

Referring to FIG. 2, FIG. 2 is another ultrasonic ablation catheter according to an embodiment of the present disclosure. A difference between this embodiment and the first embodiment lies in that a specific structure of the resilient stent 3 (a mesh-shaped structure) in this embodiment is different from that of the resilient stent 3 in the first embodiment.

The following describes the difference of the resilient stent 3 (the mesh-shaped structure) in this embodiment in detail:

In this embodiment, the resilient stent 3 (the mesh-shaped structure) is formed into a mesh shape by intertwining a plurality of stent threads 31. Specifically, in this embodiment, the plurality of stent threads 31 may be evenly grouped into two groups, stent threads 31 in a same group are parallel to each other, and the two groups of stent threads 31 form a set angle (for example: 90°), so that the two groups of stent threads 31 are braided and formed. It can be easily understood that, in other embodiments, the resilient stent 3 may be braided according to needs, to form different mesh-shaped structures.

Apart from the foregoing structure, the remaining structures in this embodiment are all the same as those in the first embodiment. Details are not described herein again.

### Third Embodiment

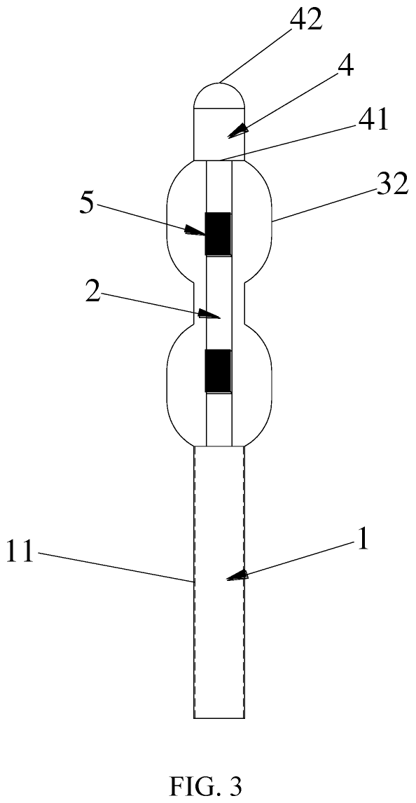

Referring to FIG. 3, FIG. 3 is another ultrasonic ablation catheter according to an embodiment of the present disclosure. A difference between this embodiment and the first embodiment lies in that a specific structure of the resilient stent 3 (a balloon-shaped structure) in this embodiment is different from that of the resilient stent 3 in the first embodiment.

The following describes the difference of the resilient stent 3 (the balloon-shaped structure) in this embodiment in detail:

In this embodiment, the resilient stent 3 (the balloon-shaped structure) includes a plurality of wrap parts 32. The wrap parts 32 are approximately ball-shaped and may be sized according to diameters of blood vessel tissue to be entered. Correspondingly, a plurality of ultrasonic transducers 5 are mounted on the central wire 2, and the plurality of wrap parts 32 are in a one-to-one mapping relationship with the plurality of ultrasonic transducers 5, so that the wrap parts 32 are respectively wrapped around outer sides of the ultrasonic transducers 5. Therefore, the ultrasonic transducers 5 wrapped by the plurality of wrap parts 32 can be isolated thereby, to avoid damage to a blood vessel wall caused by the ultrasonic transducers 5.

Apart from the foregoing structure, the remaining structures in this embodiment are all the same as those in the first embodiment. Details are not described herein again.

### Fourth Embodiment

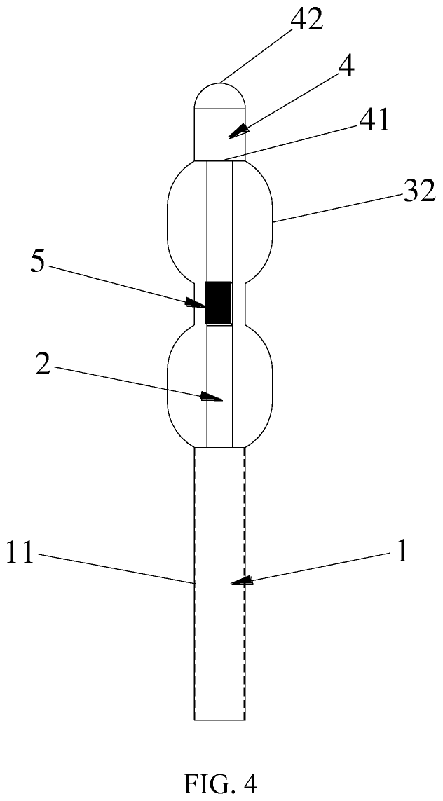

Referring to FIG. 4, FIG. 4 is another ultrasonic ablation catheter according to an embodiment of the present disclosure. A difference between this embodiment and the first embodiment lies in that a specific structure of the resilient stent 3 (a balloon-shaped structure) in this embodiment is different from that of the resilient stent 3 in the first embodiment.

The following describes the difference of the resilient stent 3 (the balloon-shaped structure) in this embodiment in detail:

In this embodiment, the resilient stent 3 (the balloon-shaped structure) includes a plurality of wrap parts 32. The wrap parts 32 are approximately ball-shaped and may be sized according to diameters of blood vessel tissue to be entered. Correspondingly, a plurality of ultrasonic transducers 5 are mounted on the central wire 2, and the plurality of wrap parts 32 and the plurality of ultrasonic transducers 5 are arranged crosswise along the axial direction of the central cavity tube 1, for any two adjacent ultrasonic transducers 5 to have one wrap part 32 therebetween. Therefore, contact between the ultrasonic transducer 5 and the blood vessel tissue can be blocked, and transfer of ultrasonic waves can be facilitated.

Apart from the foregoing structure, the remaining structures in this embodiment are all the same as those in the first embodiment. Details are not described herein again.

### Fifth Embodiment

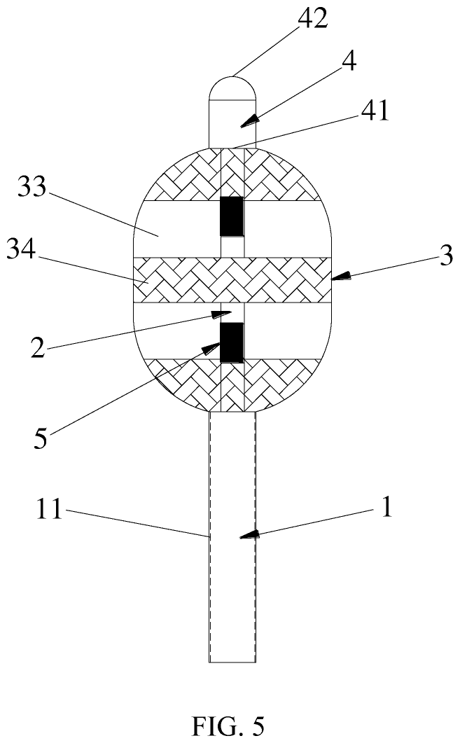

Referring to FIG. 5, FIG. 5 is another ultrasonic ablation catheter according to an embodiment of the present disclosure. A difference between this embodiment and the first embodiment lies in that a specific structure of the resilient stent 3 in this embodiment is different from that of the resilient stent 3 in the first embodiment.

The following describes the difference of the resilient stent 3 in this embodiment in detail:
in this embodiment, the resilient stent 3 is provided with a transparent refractive region 33 and an opaque closed region 34, and the transparent refractive region 33 corresponds to the ultrasonic transducer 5, to refract ultrasonic waves emitted by the ultrasonic transducer 5. Specifically, the resilient stent 3 may be formed by manufacturing transparent circles having different diameters by a transparent material (for example: PE or PE), manufacturing opaque circles having different diameters by an opaque material (for example: black silicon), then crosswise arranging the plurality of transparent circles having different diameters and the plurality of opaque circles having different diameters, and adhering adjacent transparent circles and opaque circles, and therefore the resilient stent 3 having the transparent refractive region 33 and the opaque closed region 34 is formed. Therefore, ultrasonic waves can be refracted by using the transparent refractive region 33, to adjust a direction of transfer of the ultrasonic waves, so that the ultrasonic waves can be refracted to a region that cannot be reached in a normal case, thereby improving the applicability of the ultrasonic ablation catheter.

It may be understood that when the ultrasonic waves are refracted, a specific position after the refraction has a relationship with a material, a width, and a thickness of the transparent refractive region 33, and a relative position between the transparent refractive region 33 and the ultrasonic transducer 5. In specific use, a most appropriate material, position, and size may be selected according to actual needs.

Apart from the foregoing structure, the remaining structures in this embodiment are all the same as those in the first embodiment. Details are not described herein again.

### Sixth Embodiment

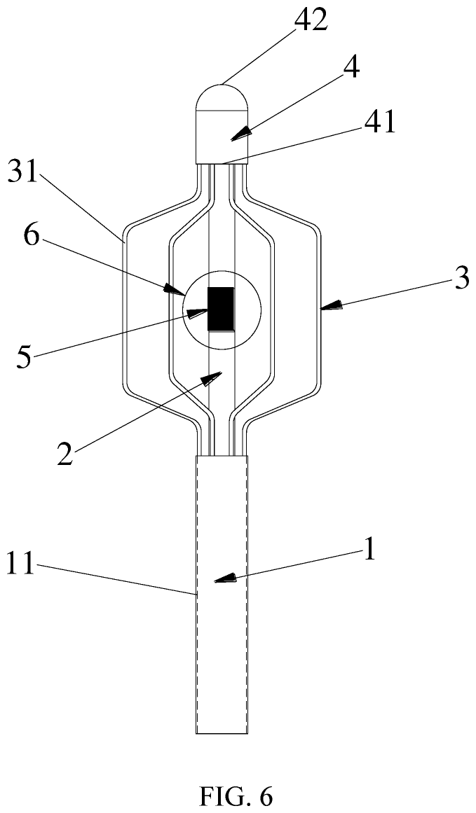

Referring to FIG. 6, FIG. 6 is another ultrasonic ablation catheter according to an embodiment of the present disclosure. A difference between this embodiment and the first embodiment lies in that a balloon 6 is further wrapped around an outer side of the ultrasonic transducer 5 in this embodiment, and the balloon 6 is arranged between the resilient stent 3 and the ultrasonic transducer 5.

Specifically, in this embodiment, the balloon 6 is wrapped around the outer side of the ultrasonic transducer 5, and the second end of the cooling pipe is enabled to extend into the balloon 6, so that cold saltwater or a cold gas can be introduced into the balloon 6 through the cooling pipe, to further cool the ultrasonic transducer 5 more conveniently.

Apart from the foregoing structure, the remaining structures in this embodiment are all the same as those in the first embodiment. Details are not described herein again.

### Seventh Embodiment

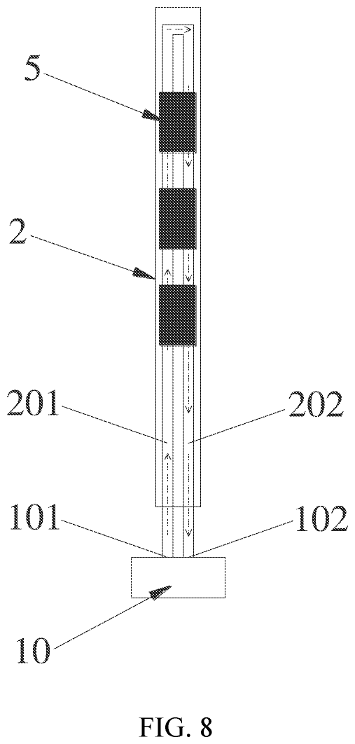

Referring to FIG. 8, FIG. 8 is another ultrasonic ablation catheter according to an embodiment of the present disclosure. Compared with the first embodiment, a difference between this embodiment and the first embodiment lies in the differences in the structures of the ultrasonic transducer 5 and the central wire 2.

Specifically, a first channel 201 and a second channel 202 communicated with each other are provided in the central wire 2. In addition, in this embodiment, both the first channel 201 and the second channel 202 pass through a bottom portion of the central wire 2 and do not pass through a top portion of the central wire 2. The first channel 201 is configured to be communicated with a liquid outlet 101 of a cold medium supply apparatus 10, and the second channel 202 is configured to be communicated with a liquid inlet 102 of the cold medium supply apparatus 10, to jointly form a cooling circulation loop (the loop shown by the dashed arrow in FIG. 7). A cold medium is periodically introduced into the cooling circulation loop, and the ultrasonic transducer 5 can be cooled by circulating flow of the cold medium in the central wire 2, to further avoid damage caused to a blood vessel wall by a high-temperature ultrasonic transducer 5, thereby improving the safety of an ablation operation. It may be understood that, in this embodiment, the cold medium periodically circulates only in the cooling circulation loop, and does not leak out to a patient body, so as to use internal cooling to avoid interference to the operation.

Moreover, in this embodiment, a width of the ultrasonic transducer 5 is slightly less than a diameter of the central wire 2. Different from the surround-type mounting mode in the first embodiment, the one or more ultrasonic transducers 5 in this embodiment are mounted on the central wire 2 by pasting and/or embedding. During specific use, the ultrasonic transducer 5 covers only a part of the central wire 2. Therefore, after the central wire 2 extends into a target site to be ablated, small-range ablation can be performed by directly using the ultrasonic transducer 5. On the basis of this, the central wire 2 may rotate in 360°, to drive the ultrasonic transducers 5 to rotate in 360°, so as to perform a large-range ablation. Therefore, the ultrasonic ablation catheter may be enabled to perform partial ablation or 360° ablation, to adapt to different ablation needs by using different ablation modes.

Apart from the foregoing structure, the remaining structures in this embodiment are all the same as those in the first embodiment. Details are not described herein again.

### Eighth Embodiment

An embodiment of the present disclosure further provides an ultrasonic ablation device, including any one of the ultrasonic ablation catheters in the first embodiment to the seventh embodiment.

Specifically, in addition to the ultrasonic ablation catheter included, the ultrasonic ablation device may further include a control handle and a control host that are connected to the ultrasonic ablation catheter. The control handle is operated by an operator and may perform pulling and pushing operations on the central wire 2 and the resilient stent 3, to switch working forms of the central wire 2 and the resilient stent 3. The control host is configured to perform temperature control, energy control, and the like on the ultrasonic transducer 5.

In addition, the ultrasonic ablation device may further be integrated with functions such as three-dimensional plotting, distinguishing a density of tissue, crushing tissue (a plaque, a clot, or the like), and ultrasonic contrast, so as to complete an ultrasonic ablation operation in coordination with the ultrasonic ablation catheter.

Compared with the existing technology, according to the ultrasonic ablation catheter and device provided in the present disclosure, controllable ablation is performed by using the ultrasonic transducer mounted on the central wire, so as to improve the ablation effect on pathological tissue. In addition, the resilient stent in the expanded state may distract a blood vessel, to prevent the ultrasonic transducer from being in contact with a blood vessel wall, thereby preventing damage to the blood vessel wall. Besides, the ultrasonic transducer may alternatively be cooled by infusing cold saltwater or a cold gas, thereby further improving the safety of an ablation operation.

The foregoing describes the ultrasonic ablation catheter and the ultrasonic ablation device provided in the present disclosure in detail. Any obvious change made by a person of ordinary skill in the art to the present disclosure without departing from the essence of the present disclosure shall constitute a violation of the patent right of the present disclosure and shall take corresponding legal responsibility.

1. An ultrasonic ablation catheter, comprising:
a central cavity tube, a central hole being provided along an axial direction of the central cavity tube;
a central wire, the central wire being provided with at least one ultrasonic transducer, and a first end of the central wire movably passing through the central hole, to switch between a contracted state and an extended state; and
a resilient stent, the resilient stent being preformed into a set shape and being nested on the central wire, and a first end of the resilient stent movably passing through the central hole, to switch between a compressed state and an expanded state, wherein
when the central wire is in the contracted state, the ultrasonic transducer and a second end of the resilient stent are accommodated in the central cavity tube together with a second end of the central wire, for the resilient stent to be in the compressed state;
and when the central wire is in the extended state, the ultrasonic transducer and the second end of the resilient stent extend out of the central cavity tube together with the second end of the central wire, for the resilient stent to be in the expanded state and restore to the set shape.

2. The ultrasonic ablation catheter according to claim 1, wherein:
the resilient stent is formed into a mesh shape by intertwining a plurality of stent threads.

3. The ultrasonic ablation catheter according to claim 1, wherein:
the resilient stent comprises a plurality of wrap parts, a plurality of ultrasonic transducers are mounted on the central wire, the wrap parts are in a one-to-one mapping relationship with the ultrasonic transducers, and the wrap parts are respectively wrapped around outer sides of the ultrasonic transducers.

4. The ultrasonic ablation catheter according to claim 1, wherein:
the resilient stent comprises a plurality of wrap parts, a plurality of ultrasonic transducers are mounted on the central wire, and the plurality of wrap parts and the plurality of ultrasonic transducers are arranged crosswise along the axial direction of the central cavity tube, for any two adjacent ultrasonic transducers to have one of the wrap parts therebetween.

5. The ultrasonic ablation catheter according to claim 1, wherein:
a balloon is further wrapped around an outer side of the ultrasonic transducer, and the balloon is arranged between the resilient stent and the ultrasonic transducer.

6. The ultrasonic ablation catheter according to claim 1, wherein:
a cooling pipe is disposed in the central hole, a first end of the cooling pipe is configured to be connected to a cold medium supply apparatus, and a second end of the cooling pipe extends out of the central cavity tube, to correspond to a position of the ultrasonic transducer when the central wire is in the extended state.

7. The ultrasonic ablation catheter according to claim 1, wherein:
a first channel and a second channel communicated with each other are provided in the central wire, the first channel is configured to be communicated with a liquid outlet of the cold medium supply apparatus, and the second channel is configured to be communicated with a liquid inlet of the cold medium supply apparatus, to jointly form a cooling circulation loop; and a cold medium periodically flows in the cooling circulation loop, to cool the ultrasonic transducer.

8. The ultrasonic ablation catheter according to claim 7, wherein:
the ultrasonic transducer is mounted on an outer side of the central wire by pasting and/or embedding, and a width of the ultrasonic transducer is less than a diameter of the central wire, to partially cover the central wire; and the central wire is capable of rotating around an axial direction of the central wire in 360°, to drive the ultrasonic transducer to rotate in 360°.

9. The ultrasonic ablation catheter according to claim 1, further comprising a connecting member, the connecting member being connected and fixed to the second end of the resilient stent and the second end of the central wire.

10. The ultrasonic ablation catheter according to claim 1, wherein:
the resilient stent is provided with a transparent refractive region and an opaque closed region, and the transparent refractive region corresponds to the ultrasonic transducer, to refract ultrasonic waves emitted by the ultrasonic transducer.

11. An ultrasonic ablation device, comprising the ultrasonic ablation catheter according to claim 1.
